(19) United States
(12) Patent Application Publication
(10) Pub. No.: US 2025/0170309 A1
(43) Pub. Date: May 29, 2025

(54) DEGRADABLE PARTICLES COMPRISING HIGH LEVELS OF RAPAMYCIN

(71) Applicant: DSM IP Assets B.V., Heerlen (NL)

(72) Inventors: Matilde PUTTI, Echt (NL); Megan HOEN, Echt (NL); Gueorgui MIHOV, Echt (NL); Jérôme George Jozeph Louis LEBOUILLE, Echt (NL)

(21) Appl. No.: 18/834,392

(22) PCT Filed: Jan. 27, 2023
(86) PCT No.: PCT/EP2023/052074

§ 371 Date: Jul. 30, 2024

Related U.S. Application Data
(60) Provisional application No. 63/305,293, filed on Feb. 1, 2022.

(30) Foreign Application Priority Data
Mar. 1, 2022 (EP) 22159350.2

(51) Int. Cl. A61L 31/10; A61L 31/16

(52) U.S. Cl. CPC A61L 31/10; A61L 31/16; A61L 2300/416; A61L 2300/622

(57) ABSTRACT

Disclosed are drug delivery devices, coatings, and microparticles that achieve sufficiently small particle sizes with high loading of Rapamycin. In an embodiment, a balloon comprises a coating on its exterior surface, the coating comprising microparticles having a Dv90 particle size of from 1 to 11 μm, as measured with static laser scattering, the microparticles comprising from 35 to 50 wt % of rapamycin, based on the total weight of the microparticles, and a random copolymer according to Formula (I).

### CROSS-REFERENCE TO RELATED APPLICATIONS

This application is an international application claiming priority to U.S. Provisional Application No. 63/305,293, filed 1 Feb. 2022, and European Patent Application No. EP22159350.2, filed 1 Mar. 2022, the entire contents of each of which is hereby incorporated by reference in its entirety.

### FIELD

The disclosed inventions pertain to degradable particles comprising rapamycin, methods of forming such particles, medical devices comprising such particles, and methods of treating mammals suffering from various conditions using such particles.

### BACKGROUND

It is known that rapamycin, also known as sirolimus, may be coated onto a balloon catheter in order to address or improve various patient outcomes. The rapamycin is typically encapsulated or dispersed in various materials and coated on the balloon in particle format. Inflation of the balloon causes the coating to contact the blood vessel wall and deliver the particles. The delivery of rapamycin occurs when it leaches out of the particles or when the particles degrade. Drug delivery devices comprising degradable polymers may be preferred because they may not require a separate procedure to remove the polymer after the bioactive agent is depleted.

The particles used must be sufficiently small in order to be useable as a coating on a balloon catheter. This is because it is possible that particles enter the blood stream. In that event, smaller particles are less likely to cause an adverse effect. A particle size of 10 μm or less is desired.

It is challenging to form particles comprising rapamycin and having a particle size of 10 μm or less while achieving sufficiently high rapamycin loading. Various challenges are particle formation, particle size distribution, particle agglomeration, sufficient release duration at acceptable quantities of rapamycin, and achieving sufficient therapeutic dose of rapamycin throughout the desired therapeutic window.

### SUMMARY

Particles that enable surprisingly high loading of rapamycin at the required particle size have been discovered. Such particles have a particle size Dv90 of 11 μm or less. Such particles may provide an extended and sufficient release of rapamycin within the necessary particle size window, thereby improving patient safety and treatment efficacy.

The disclosed microparticles, coatings, methods, and drug delivery devices may achieve benefits in the release of rapamycin, such as greater release duration, more uniform daily dose delivery, or a more desirable amount of daily dose, particle formation, particle size distribution, particle agglomeration, sufficient release duration at acceptable quantities of rapamycin, and achieving sufficient therapeutic dose of rapamycin throughout the desired therapeutic window.

### DETAILED DESCRIPTION

The invention employs a random copolymer is according to Formula I:

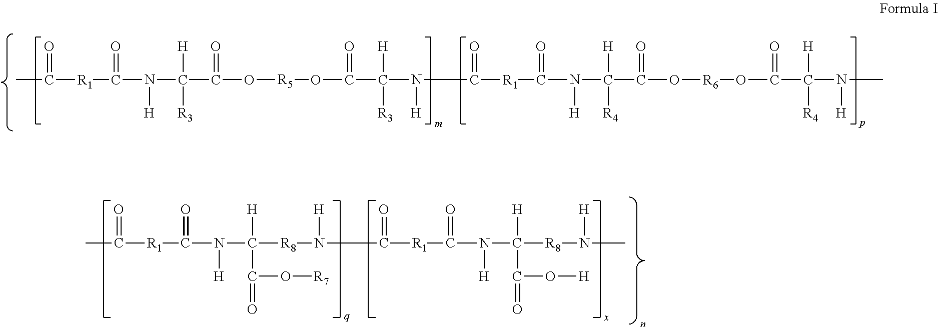

wherein
m, p, q, and x represent the equivalents of the corresponding units in the random copolymer and m+p+q+x=1;
m is from 0 to 0.8, p is from 0 to 0.8, m+p is from is from 0.5 to 0.9, q is from 0.05 to 0.25, x is from 0.05 to 0.25, and q:x is from 9:1 to 1:4;
n is from 5 to 300;
R¹ is C₂-C₂₀ alkylene;
R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—,

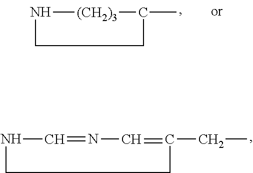

wherein R³ and R⁴ may be the same or different;
R⁶ is according to Formula II or Formula III;

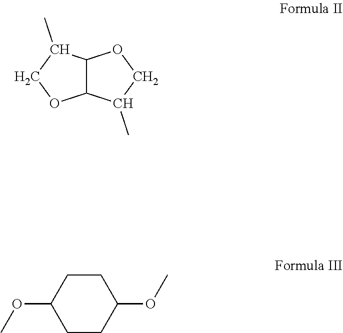

R⁷ is (C₆-C₁₀)aryl(C₁-C₆)alkylene; and
R⁸ is C₃-C₈ alkylene.

As used herein, the term “alkyl” means a monovalent straight or branched chain hydrocarbon group including methyl, ethyl, n-propyl, isopropyl, n-butyl, isobutyl, tert-butyl, n-hexyl, and the like.

As used herein, the term “alkylene” means a divalent branched or unbranched hydrocarbon chain such as —CH₂—, —(CH₂)₂—, —(CH₂)₃—, —(CH₂)₄—, —(CH₂)₅—, and the like.

As used herein, the term “alkenyl” means a monovalent straight or branched chain hydrocarbon group containing at least one unsaturated bond in the main chain or in a side chain.

As used herein, “alkenylene”, means a divalent branched or unbranched hydrocarbon chain containing at least one unsaturated bond in the main chain or in a side chain.

As used herein, “alkynyl”, means a straight or branched hydrocarbon chain having at least one carbon-carbon triple bond.

As used herein, “aryl” means a phenyl radical or an ortho-fused bicyclic carbocyclic radical having about nine to ten ring atoms, in which at least one ring is aromatic. Examples of aryl include, but are not limited to, phenyl, naphthyl, and nitrophenyl.

As used herein, “biodegradable” means a material which is capable of being completely or substantially degraded or eroded when exposed to an in vivo environment. A polymer is capable of being degraded or eroded when it can be gradually broken-down, resorbed, absorbed and/or eliminated by, for example, hydrolysis, enzymolysis, oxidation, metabolic processes, bulk or surface erosion, and the like.

As used herein, “random copolymer” means a copolymer wherein two or more individual polymer units are distributed randomly throughout the copolymer. In accordance with Formula I, each of the units m, p, q, and x are randomly distributed throughout the copolymer.

In an embodiment, m is from 0.10, 0.15, 0.20, or 0.25 to 0.75, 0.70, 0.65, 0.60, 0.55, 0.50, 0.45, 0.40, or 0.35. In an embodiment, p is from 0.10, 0.20, 0.30, 0.35, or 0.40 to 0.75, 0.70, 0.65, 0.60, 0.55, 0.50, or 0.45. In an embodiment, p is greater than or equal to m. In an embodiment, m and p are greater than zero. m:p is from 2:1, 1:1, or 2:3 to 1:5, 1:4, 1:3, or 1:2.

In an embodiment, the q is from 0.05, 0.10, 0.12, 0.14, 0.15, 0.16, 0.17, 0.18, 0.19, or 0.2 to 0.25, 0.23, 0.21, 0.20, 0.19, 0.18, 0.17, 0.16, or 0.15. In an embodiment, x is from 0.05, 0.06, 0.07, 0.08, 0.09, or 0.10 to 0.25, 0.20, 0.15, 0.14, 0.13, 0.12, 0.11, or 0.10. In an embodiment, the ratio q:x is from 9:1, 8:1, 7:1, 6:1, 5:1, 4:1 or 3:1 to 1:4, 1:3, 1:2, 1:1, 2:1, or 3:1.

In an embodiment, m is about 0.3, p is about 0.45, q is about 0.19, and x is about 0.06. In an embodiment, n is from 5, 10, 15, 20, 25, 30, 35, 40, 45, 50, 55, 60, 65, 70, or 75 to 300, 290, 280, 270, 260, 250, 240, 230, 220, 210, 200, 190, 180, 170, 160, or 150. In an embodiment, the random copolymer of Formula I has a number average molecular weight (Mn) of at least 15,000 g/mol, at least 20,000 g/mol, at least 25,000 g/mol, at least 30,000 g/mol, or at least 35,000 g/mol. In an embodiment, the random copolymer of Formula I has a Mn of at most 250,000 g/mol, at most 225,000 g/mol, at most 200,000 g/mol, at most 175,000 g/mol, at most 150,000 g/mol, at most 125,000 g/mol, at most 100,000 g/mol, or at most 75,000 g/mol. Mn is measured via GPC in THF with polystyrene as standard.

In an embodiment, R³ is hydrogen, (C₁-C₆)alkyl, CH₃—CH₂—CH(CH₃)—, (CH₃)₂CH—CH₂—, Ph-CH₂—, or (CH₃)₂CH—. R⁴ is hydrogen, (C₁-C₆)alkyl, CH₃—CH₂—CH(CH₃)—, (CH₃)₂CH—CH₂—, Ph-CH₂—, or (CH₃)₂CH—. In an embodiment, R³ and R⁴ are the same. In an embodiment, R⁷ is C₆aryl-CH₂— (i.e. benzyl). In an embodiment, R⁸ is —(CH₂)₄—.

Polyesteramide random copolymers are synthesized by adapting a procedure known in the art. R. Katsarava, V. Beridze, N. Arabuli, D. Kharadze, C. C. Chu, C. Y. Won J Polym Sci A: Polym Chem 37:391-407, 1999. Briefly, the polymers are prepared via solution polycondensation of di-p-toluenesulfonic or hydrochloric acid salts of bis-(α-amino acid) α,ω-diol diesters, lysine benzyl ester, lysine, and/or di-N-hydroxysuccinimide ester of sebacic acid in anhydrous DMSO. Typically, the salts are converted to free amines by addition of triethylamine and these amines are further reacted with the di-acid derivative. The usage of pre-activated acid in the reaction allows polymerization at relatively low temperature, such as 65° C., affording side-product free polycondensates and predictable degradation products. Subsequently, the obtained reaction mixture is purified via a water precipitation followed by an organic precipitation and filtration. Drying under reduced pressure yields the polyesteramide random copolymer.

For example, such polymers may be prepared by reacting lysine, lysine benzyl ester, and hexahydrofuro[3,2-b]furan-3,6-diyl bis(2-amino-4-methylpentanoate) with di-N-hydroxysuccinimide ester activated sebacic acid in DMSO for 24 hours. The polymer is then isolated from the reaction mixture in two precipitation steps and characterized by means of proton NMR and THF-based GPC relative to polystyrene standards.

Embodiments of the invention comprise microparticles or films formed from microparticles. In an embodiment, the microparticles comprise rapamycin dispersed throughout the random copolymer according to Formula I, such as a mixture of Rapamycin and the random copolymer. In an embodiment, the microparticles comprise from 35 to 50 wt % of rapamycin, based on the total weight of the microparticles. In an embodiment, the microparticles comprise from 35 to 45 wt % Rapamycin. In an embodiment, the microparticles consist of Rapamycin and the random copolymer according to Formula I.

In an embodiment, the microparticles are formed via an oil-in-water emulsion technique as described herein. In an embodiment, the oil phase comprises from 1 to 2.5 wt % Rapamycin, 1.5 to 2.5 wt % of a random copolymer according to Formula I, and from 95 to 97.5 wt % of a chlorinated methane solvent. In an embodiment, the oil phase comprises from 1.3 to 2.1 wt % of Rapamycin, from 1.5 to 2.5 wt % of the random copolymer according to Formula I, and from 95.5 to 97.2 wt % of the chlorinated methane solvent.

The water phase comprises water and an emulsifier. In an embodiment, the emulsifier is present in at least 0.1, 0.25, or 0.4 wt % of the water phase. In an embodiment, the emulsifier is present in at most 1.0, 0.8, or 0.6 wt % of the water phase. In an embodiment, the emulsifier is polyvinyl alcohol (PVA).

In an embodiment, the oil phase further comprises an antioxidant, such as butylated hydroxytoluene (BHT). In embodiment, the chlorinated methane solvent is selected from the group consisting of chloroform, dichloromethane, and mixtures thereof.

The microparticles have a Dv90 particle size of from 1 to 11 μm. In an embodiment, the microparticles have a Dv90 particle size of from 1, 2, 3, 4, 5, 6, 7, or 8 μm. In an embodiment, the microparticles have a Dv90 particle size of at most 11, 10, 9, or 8 μm. Dv90 particle size is measured by static light laser scattering using a Malvern Mastersizer equipped with an aqueous medium sample dispersion chamber.

In an embodiment, a drug delivery device comprises the microparticles. In an embodiment, the drug delivery device provides for a controlled and/or extended release of rapamycin. A drug delivery device may be a pharmaceutical product or a medical device. A pharmaceutical product is a medical product that is administered to a patient and achieves its primary intended purpose through pharmacological action. A medical device is a medical instrument, apparatus, implement, machine, contrivance, implant, in vitro reagent, or other similar or related article, including a component part or accessary thereof, that does not achieve its primary intended purpose through pharmacological action.

The drug delivery device may take various forms. In an embodiment, the drug delivery device comprises the microparticles. In an embodiment, the drug delivery device comprises a coating comprising the microparticles. The coating is formed on the surface of the device, such as an exterior surface of a stent or balloon. The microparticles may have special utility for forming a device that may deliver rapamycin to a blood vessel wall via the inflation of a balloon. Accordingly, in an embodiment, a coated balloon comprises the microparticles on its exterior surface.

A coating may be formed in various ways. In an embodiment, a coating formulation is prepared comprising a matrix and the microparticles. The coating formulation is then applied to the surface of a medical device and cured, yielding the coated medical device. In an embodiment, the microparticles are adhered to the surface of the medical device, for instance, by contacting a surface of the medical device with a quantity of the microparticles. The microparticles may adhere to the surface due to the natural tackiness of the surface or the microparticles, optionally activated with, for example, water, or by the use of an adhesive present on the surface.

The drug delivery devices comprising the random copolymers may be used in the medical field especially in the cardiovascular field.

The Examples below further elucidate embodiments of the invention, but of course, should not be construed as in any way limiting the scope of the claims.

### EXAMPLES

### Measurement Methods

### Molecular Weight

Mn was measured via GPC using THE as the mobile phase on dried samples. Molecular weights are relative to polystyrene standards.

### Particle Size

Particle size distribution was measured by static laser scattering using a Malvern MasterSizer equipped with an aqueous medium sample dispersion chamber. Microparticles are initially dispersed in 2 mL PBS containing 0.04% polysorbate-80 at 10 mg/mL. The dispersed particles are then injected in the Hydro 2000S dispersion chamber containing MilliQ water under 1500 rpm stirring. Sonication is applied during measurement. Each measurement is an average of triplicate measurement per run. Particle size results are calculated by the instrument software from scattered light intensity according to the Mie theory, applying the refractive index of water for the dispersant (1.33) and 1.5 as the refractive index of the material. Particle sizes are expressed as volume-weighed averages.

### Rapamycin Content

Rapamycin content in the microparticles is measured by dissolving 10 mg of formulation in absolute Ethanol by stirring overnight. The ethanol solution containing Rapamycin was filtered and Rapamycin quantified by Reverse Phase (RP)-HPLC-UV by extrapolation from a calibration curve in a suitable range of concentrations.

### Preparation of Copolymers Used in the Examples

PEA III X25 is a random copolymer according to Formula IV and may be obtained as follows.

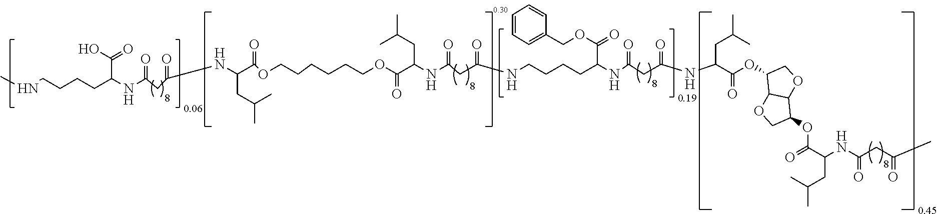

Triethylamine (31 mL, 0.222 mole) and DMSO (54 mL, 0.76 mole) are added to a mixture of di-N-hydroxysuccinimide ester of sebacic acid (Di-NHS-sebacic acid) (39.336 g, 0.099 mole), L-leucine-(DAS)-2TosOH (32.876 g, 0.045 mole), L-leucine (6)-2TosOH (21.062 g, 0.030 mole), L-lysine·2HCl (1.396 g, 0.006 mole) and L-lysine (Bz)-2TosOH (4.235 g, 0.018 mole) in a nitrogen flushed 500 mL round bottomed flask equipped with an overhead stirrer at room temperature. The subsequent mixture is heated to 60° C. to allow the reaction to proceed and monitored by GPC analysis in THF. After 36 hours a stable molecular weight is obtained. The reaction mixture is diluted with 250 mL DMSO and is allowed to cool to room temperature. At room temperature acetic anhydride (1.89 mL, 0.0199 mole) is added to acylate the amino functional end groups of the polymer. Next, the mixture is stirred at room temperature for 24 hours.

The obtained crude polymer mixture is precipitated in water at a 10:1 ratio (water:reaction mixture). The polymer is collected and dissolved in ethanol (500 mL, 8.57 mole) and then precipitated a second time. The polymer is again dissolved in ethanol (500 mL, 8.57 mole) and precipitated in ethylacetate (5000 mL, 50.91 mole) by drop wise addition to a stirring solution. The precipitated polymer is washed with ethylacetate (100 mL, 1.00 mole), the supernatant is removed, and the precipitate is washed again with ethylacetate (100 mL, 1.00 mole). After the removal of the supernatant, the precipitate is dried and dissolved in ethanol (500 mL, 8.57 mole), and filtered over a 0.2 μm PTFE membrane filter. The filtered polymer solution is dried under reduced pressure at 65° C. The typical yield is 75%, Mn is typically in the range of 45-70 kDa (Gel Permeation Chromatography (GPC) in THE relative to polystyrene standards).

### Preparation of Microparticles

The oil phase was prepared by dissolving the polymer, Rapamycin, and optionally stabilizer 2,6-bis(1,1-dimethylethyl)-4-methylphenol (BHT), in chloroform in a glass container at the stated amounts. Dissolution was carried out overnight under magnetic stirring.

The water phase, or continuous phase, comprises 0.5% w/w Poly(vinyl alcohol) (PVA) (9-10 k g/mol, 80% hydrolyzed) and 99.5% w/w demineralized water. The PVA was dissolved at room temperature in water under stirring for at least 2 hours.

The emulsification was carried out in 50 mL glass beakers using an Ultra-Thurrax IKA T25 stirring head, equipped with a S25N-10G dispersion tool. 20 mL of water phase are poured into the beaker and the stirring speed is set at 10200 rpm. 4 mL of oil phase are injected all at once with a 21G needle. After complete injection, the emulsification is carried out for 3 minutes. The obtained emulsion was allowed to degas under magnetic stirring overnight to let the organic solvent evaporate.

After overnight hardening, the particles were isolated by centrifugation at 4000 g. The obtained pellet was washed three times with 5 mL of an aqueous solution containing 0.04% w/w Tween-80 in demineralized water by consecutive resuspension/centrifugation cycles. The washed particles were resuspended in 5 mL of demineralized water and lyophilized to obtain a white, dry powder formulation.

### Example 1—Encapsulation Efficiency of Rapamycin

Eight oil phase compositions are prepared. Microparticles were prepared by emulsification as described previously. Rapamycin content and particle size were measured. The compositions, measured Rapamycin content, and measured particle size are shown in Table 1.

TABLE 1
Oil-phase Compositions and Results

| | Component amount [wt %] | | | | Dv10/ | |
|---|---|---|---|---|---|---|
| | PEA | | | | Dv50/ | Rapamycin |
| | III | | | | Dv90 | Content |
| | X25 | Rapamycin | BHT | Chloroform | [μm] | [wt %] |
| 1 | 2.09 | 1.59 | 0.0016 | 96.32 | 3/6/12 | 41 |
| 2 | 2.00 | 1.33 | — | 96.67 | 3/6/11 | 35 |
| 3 | 2.02 | 2.03 | — | 95.94 | 2/4/10 | 45 |
| 4 | 4.51 | 3.02 | — | 92.46 | 2/7/15 | 37 |
| 5 | 3.22 | 2.42 | — | 94.35 | 4/9/19 | 39 |
| 6 | 3.38 | 2.14 | — | 94.36 | 5/11/28 | 35 |
| 7 | 3.62 | 2.35 | — | 94.02 | 7/14/25 | 35 |
| 8 | 1.36 | 0.86 | — | 97.77 | 3/6/15 | 33 |

Experiments 2 and 3 successfully meet the required Dv90 particle size and rapamycin loading. The other experiments do not achieve the required Dv90 particle size.

The following non-limiting list of exemplary embodiments is included to further elucidate certain embodiments of the invention.

### Additional Description of Exemplary Embodiments

1. A balloon comprising a coating on its exterior surface, the coating comprising microparticles having a Dv90 particle size of from 1 to 11 μm, as measured with static laser scattering, the microparticles comprising from 35 to 50 wt % of rapamycin, based on the total weight of the microparticles, and a random copolymer according to Formula I:

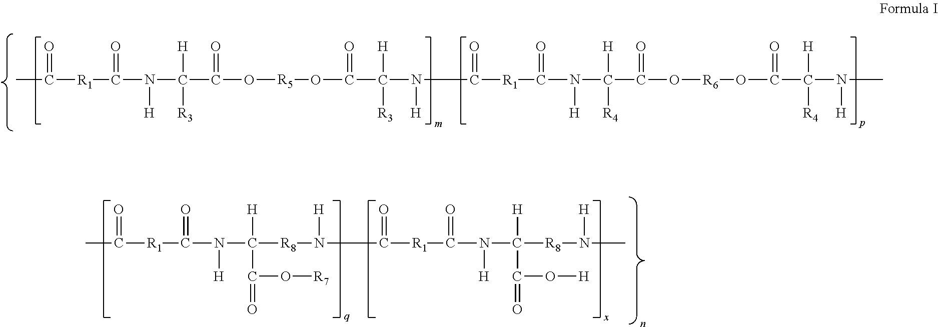

wherein
   m, p, q, and x represent the equivalents of the corresponding units in the random copolymer and m+p+q+x=1;
   m is from 0 to 0.75, p is from 0 to 0.75, m+p is from is from 0.5 to 0.9, q is from 0.05 to 0.25, x is from 0.01 to 0.25, and q:x is from 9:1 to 1:4;
   n is from 5 to 300;
   R¹ is C₂-C₂₀ alkylene;
   R³ and R⁴ are hydrogen, (C₁-C₆)alkyl, (C₂-C₆)alkenyl, (C₂-C₆)alkynyl, (C₈-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—,

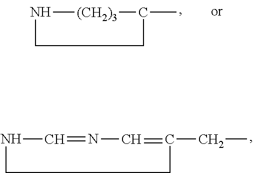

wherein R³ and R⁴ may be the same or different;
R⁶ is according to Formula II or Formula III;

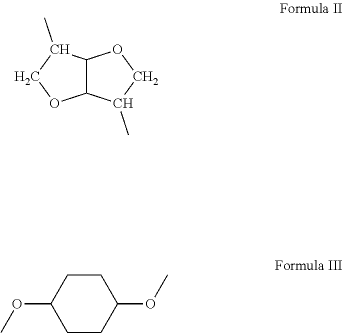

R⁷ is (C₆-C₁₀)aryl(C₁-C₆)alkylene; and
R⁸ is C₃-C₈ alkylene.
2. A method of forming a drug delivery device comprising the steps of:
   a. forming a coating on a surface of a medical device, the coating comprising microparticles having a Dv90 particle size of from 1 to 11 μm, as measured with static laser scattering, the microparticles comprising from 35 to 50 wt % of rapamycin, based on the total weight of the microparticles, and a random copolymer according to Formula I:

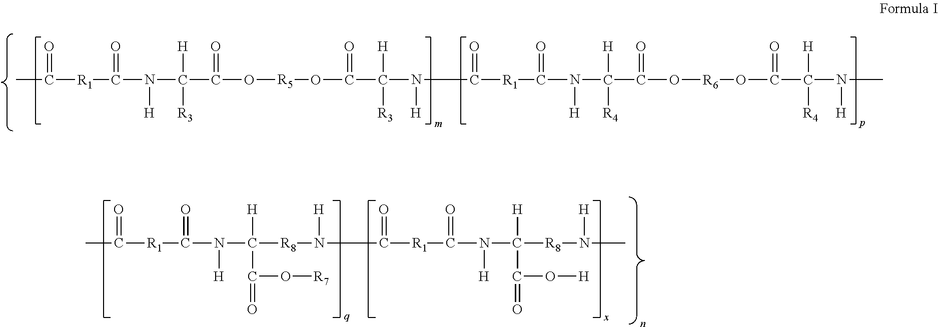

wherein
   m, p, q, and x represent the equivalents of the corresponding units in the random copolymer and m+p+q+x=1;
   m is from 0 to 0.75, p is from 0 to 0.75, m+p is from is from 0.5 to 0.9, q is from 0.05 to 0.25, x is from 0.03 to 0.25, and q:x is from 9:1 to 1:4;
   n is from 5 to 300;
   R¹ is C₂-C₂₀ alkylene;
   R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—, CH═C—CH₂—, (CH₃)₂CH—, Ph-NH—,

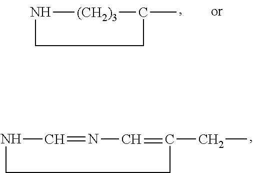

wherein R³ and R⁴ may be the same or different;
R⁶ is according to Formula II or Formula III;

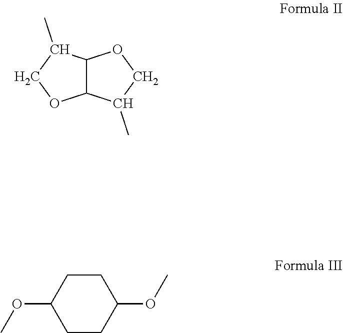

R⁷ is (C₆-C₁₀)aryl(C₁-C₆)alkylene; and
R⁸ is C₃-C₈ alkylene.
3. The method of the previous exemplary embodiment, wherein the surface is the exterior surface of a balloon.
4. The method of any of the previous two exemplary embodiments, wherein the coating comprises a matrix and the microparticles.
5. The method of any of the previous three exemplary embodiments, where the matrix is water soluble.
6. A method of forming microparticles comprising the steps of:
   a. forming an oil phase comprising from 1 to 2.5 wt % Rapamycin, 1.5 to 2.5 wt % of a random copolymer according to Formula I, and from 95 to 97.5 wt % of chloroform or dichloromethane,
   b. forming a continuous phase comprising water and from 0 to 1 wt % of an emulsifier, and
   c. adding the oil phase to continuous phase under high shear, wherein the random copolymer according to Formula I is as follows:

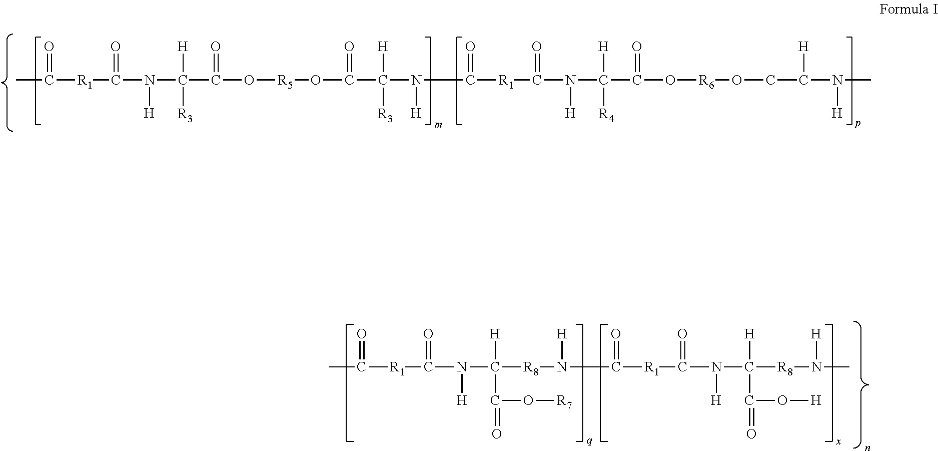

wherein
   m, p, q, and x represent the equivalents of the corresponding units in the random copolymer and m+p+q+x=1;
   m is from 0 to 0.75, p is from 0 to 0.75, m+p is from is from 0.5 to 0.9, q is from 0.05 to 0.25, x is from 0.03 to 0.25, and q:x is from 9:1 to 1:4;
   n is from 5 to 300;
   R¹ is C₂-C₂₀ alkylene;
   R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—, CH═C—CH₂—, (CH₃)₂CH—, Ph-NH—,

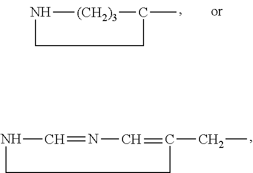

wherein R³ and R⁴ may be the same or different;
R⁶ is according to Formula II or Formula III;

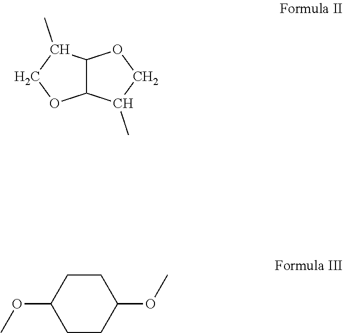

R⁷ is (C₆-C₁₀)aryl(C₁-C₅)alkylene; and
R⁸ is C₃-C₈ alkylene.
7. The method according to the previous exemplary embodiment, wherein the oil phase further comprises an antioxidant.
8. The method according to the previous exemplary embodiment, wherein the antioxidant comprises butylated hydroxytoluene.
9. The method according to any of the three previous exemplary embodiments, wherein the emulsifier comprises a polyvinyl alcohol.
10. The method according to any of the four previous exemplary embodiments, wherein the oil-phase comprises from 1.25 to 2.1 wt % of Rapamycin, from 1.5 to 2.5 wt % of the random copolymer according to Formula I, and from 95.5 to 97.2 wt % of chloroform or dichloromethane.
11. The method according to any of the five previous exemplary embodiments, wherein the oil-phase comprises from 1.25 to 2.1 wt % of Rapamycin, from 1.9 to 2.07 wt % of the random copolymer according to Formula I, and from 95.5 to 97.0 wt % of chloroform or dichloromethane.
12. A plurality of microparticles having a Dv90 particle size of from 1 to 11 μm, as measured with static laser scattering, the microparticles consisting of from 35 to 50 wt % of rapamycin, based on the total weight of the microparticles, and a random copolymer according to Formula I:

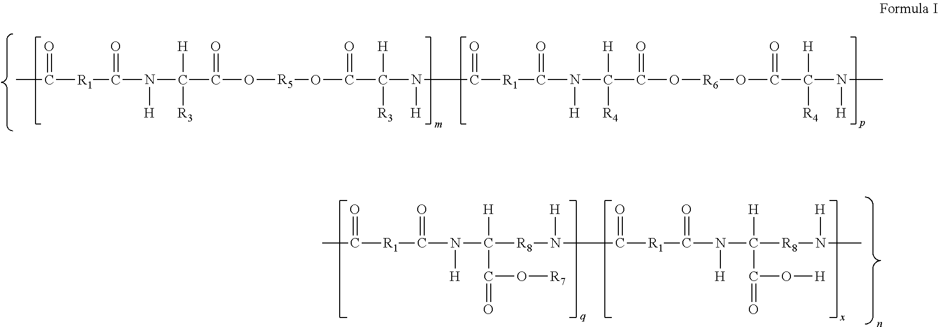

wherein
   m, p, q, and x represent the equivalents of the corresponding units in the random copolymer and m+p+q+x=1;
   m is from 0 to 0.75, p is from 0 to 0.75, m+p is from is from 0.5 to 0.9, q is from 0.05 to 0.25, x is from 0.01 to 0.25, and q:x is from 9:1 to 1:4;
   n is from 5 to 300;
   R¹ is C₂-C₂₀ alkylene;
   R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—, CH═C—CH₂—, (CH₃)₂CH—, Ph-NH—,

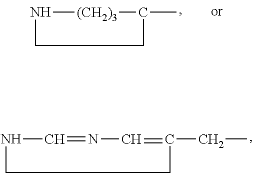

wherein R³ and R⁴ may be the same or different;
R₆ is according to Formula II or Formula III;

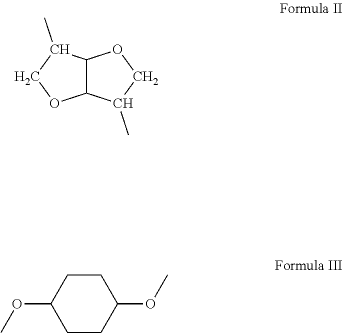

R⁷ is (C₆-C₁₀)aryl(C₁-C₆)alkylene; and
R⁸ is C₃-C₈ alkylene.
13. A coating comprising the plurality of microparticles according to the previous exemplary embodiment or formed from the method of a previous exemplary embodiment.
14. The balloon, coating, method, or microparticles of any previous exemplary embodiment, wherein m is from 0.10, 0.15, 0.20, or 0.25 to 0.75, 0.70, 0.65, 0.60, 0.55, 0.50, 0.45, 0.40, or 0.35.
15. The balloon, coating, method, or microparticles of any previous exemplary embodiment, wherein p is from 0.10, 0.20, 0.30, 0.35, or 0.40 to 0.75, 0.70, 0.65, 0.60, 0.55, 0.50, or 0.45.
16. The balloon, coating, method, or microparticles of any previous exemplary embodiment, wherein p is greater than or equal to m.
17. The balloon, coating, method, or microparticles of any previous exemplary embodiment, wherein m and p are greater than zero. m:p is from 2:1, 1:1, or 2:3 to 1:5, 1:4, 1:3, or 1:2.
18. The balloon, coating, method, or microparticles of any previous exemplary embodiment, wherein the q is from 0.05, 0.10, 0.12, 0.14, 0.15, 0.16, 0.17, 0.18, 0.19, or 0.2 to 0.25, 0.23, 0.21, 0.20, 0.19, 0.18, 0.17, 0.16, or 0.15.
19. The balloon, coating, method, or microparticles of any previous exemplary embodiment, wherein x is from 0.05, 0.06, 0.07, 0.08, 0.09, or 0.10 to 0.25, 0.20, 0.15, 0.14, 0.13, 0.12, 0.11, or 0.10.
20. The balloon, coating, method, or microparticles of any previous exemplary embodiment, wherein the ratio q:x is from 9:1, 8:1, 7:1, 6:1, 5:1, 4:1 or 3:1 to 1:4, 1:3, 1:2, 1:1, 2:1, or 3:1.
21. The balloon, coating, method, or microparticles of any previous exemplary embodiment, wherein m is 0.28 to 0.32, p is 0.43 to 0.47, q is 0.17 to 0.21, and x is 0.04 to 0.08.
22. The balloon, coating, method, or microparticles of any previous exemplary embodiment, wherein m is about 0.3, p is about 0.45, q is about 0.19, and x is about 0.06.
23. The balloon, coating, method, or microparticles of any previous exemplary embodiment, wherein n is from 5, 10, 15, 20, 25, 30, 35, 40, 45, 50, 55, 60, 65, 70, or 75 to 300, 290, 280, 270, 260, 250, 240, 230, 220, 210, 200, 190, 180, 170, 160, or 150.
24. The balloon, coating, method, or microparticles of any previous exemplary embodiment, wherein the random copolymer of Formula I has a number average molecular weight (Mn) of at least 15,000 g/mol, at least 20,000 g/mol, at least 25,000 g/mol, at least 30,000 g/mol, or at least 35,000 g/mol.
25. The balloon, coating, method, or microparticles of any previous exemplary embodiment, wherein the random copolymer of Formula I has a Mn of at most 250,000 g/mol, at most 225,000 g/mol, at most 200,000 g/mol, at most 175,000 g/mol, at most 150,000 g/mol, at most 125,000 g/mol, at most 100,000 g/mol, or at most 75,000 g/mol.
26. The balloon, coating, method, or microparticles of any previous exemplary embodiment, wherein x is from 0.02 to 0.15.
27. The balloon, coating, method, or microparticles of any previous exemplary embodiment, wherein m is from 0.10 to 0.75 and p is from 0.10 to 0.75.
28. The balloon, coating, method, or microparticles of any previous exemplary embodiment, wherein m is from 0.2 to 0.4, p is from 0.3 to 0.6, m+p is from 0.65 to 0.85, q is from 0.1 to 0.3, and x is from 0.02 to 0.15.
29. The balloon, coating, method, or microparticles of any previous exemplary embodiment, wherein m is from 0.25 to 0.35, p is from 0.40 to 0.50, m+p is from 0.70 to 0.80, q is from 0.15 to 0.25, and x is from 0.03 to 0.10.
30. The balloon, coating, method, or microparticles of any previous exemplary embodiment, wherein the ratio of q to x is from 5:1 to 1:3.
31. The balloon, coating, method, or microparticles of any previous exemplary embodiment, wherein the ratio of q to x is from 5:1 to 1:1.
32. The balloon, coating, method, or microparticles of any previous exemplary embodiment, wherein the ratio of q to x is from 4:1 to 2:1.
33. The balloon, coating, method, or microparticles of any previous exemplary embodiment, wherein n is from 50 to 200.
34. The balloon, coating, method, or microparticles of any previous exemplary embodiment, wherein R¹ is —(CH₂)₈— or —(CH₂)₄—.
35. The balloon, coating, method, or microparticles of any previous exemplary embodiment, wherein R³ is hydrogen, (C₁-C₆)alkyl, CH₃—CH₂—CH(CH₃)—, (CH₃)₂CH—CH₂—, Ph-CH₂—, or (CH₃)₂CH—.
36. The balloon, coating, method, or microparticles of any previous exemplary embodiment, wherein R⁴ is hydrogen, (C₁-C₆)alkyl, CH₃—CH₂—CH(CH₃)—, (CH₃)₂CH—CH₂—, Ph-CH₂—, or (CH₃)₂CH—.
37. The balloon, coating, method, or microparticles of any previous exemplary embodiment, wherein R³ and R⁴ are the same.
38. The balloon, coating, method, or microparticles of any previous exemplary embodiment, wherein R⁶ is according to Formula II.
39. The balloon, coating, method, or microparticles of any previous exemplary embodiment, wherein R⁷ is C₆aryl-CH₂—.
40. The balloon, coating, method, or microparticles of any previous exemplary embodiment, wherein R⁸ is —(CH₂)₄—.
41. The balloon, coating, method, or microparticles of any previous exemplary embodiment, wherein the microparticles have a Dv90 particle size of from 1, 2, 3, 4, 5, 6, 7, or 8 μm.
42. The balloon, coating, method, or microparticles of any previous exemplary embodiment, wherein the microparticles have a Dv90 particle size of at most 11, 10, 9, or 8 μm.
43. The balloon, coating, method, or microparticles of any previous exemplary embodiment, wherein the microparticles comprise from 35 to 45 wt % of rapamycin.
44. The balloon, coating, method, or microparticles wherein the microparticles comprise a mixture of the random copolymer according to Formula I and rapamycin.
45. The balloon, coating, method, or microparticles wherein the microparticles consist of a mixture of the random copolymer according to Formula I and rapamycin.
46. A dispersion of microparticles in water formed by the method of any one of the previous exemplary embodiments.
47. The dispersion of the previous exemplary embodiment, wherein the microparticles have a Dv90 particle size of from 1 to 11 μm, as measured with static laser scattering, and comprise from 35 to 45 wt % of rapamycin.
48. A method of treating a human or animal patient comprising the steps of:
   a. providing the balloon in accordance with any previous exemplary embodiment;
   b. positioning the balloon in a blood vessel of a patient;
   c. inflating the balloon and thereby transferring a quantity of the microparticles to a wall of the blood vessel; and
   d. deflating the balloon and removing the coated balloon from the blood vessel of the patient.
49. Use of the balloon according to any previous exemplary embodiment for treating an arterial disease.
50. The balloon according to any previous exemplary embodiment, for use in treating an arterial disease of a patient.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

1. A balloon comprising a coating on its exterior surface, the coating comprising microparticles having a Dv90 particle size of from 1 to 11 μm, as measured with static laser scattering, the microparticles comprising from 35 to 50 wt % of rapamycin, based on the total weight of the microparticles, and a random copolymer according to Formula I: 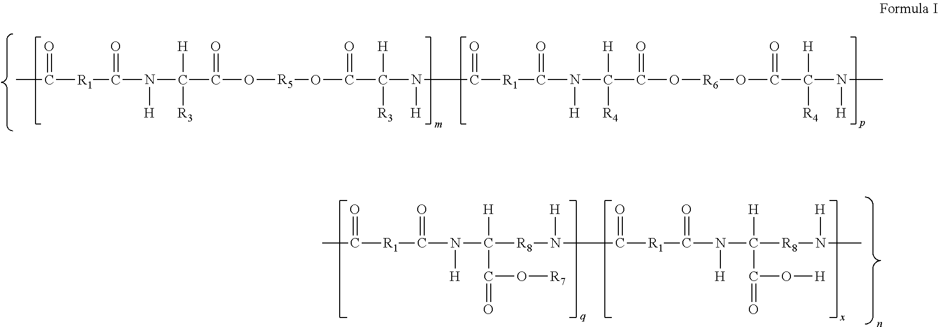
wherein
m, p, q, and x represent the equivalents of the corresponding units in the random copolymer and m+p+q+x=1;
m is from 0 to 0.75, p is from 0 to 0.75, m+p is from is from 0.5 to 0.9, q is from 0.05 to 0.25, x is from 0.01 to 0.25, and q:x is from 9:1 to 1:4;
n is from 5 to 300;
R¹ is C₂-C₂₀ alkylene;
R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—, CH═C—CH₂—, (CH₃)₂CH—, Ph-NH—, 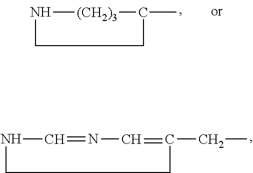
wherein R³ and R⁴ may be the same or different;
R⁶ is according to Formula II or Formula III; 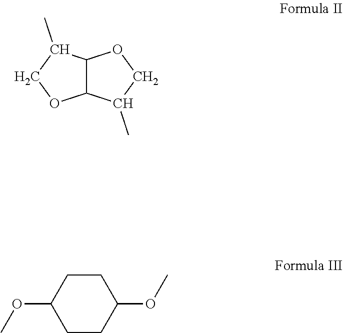
R⁷ is (C₆-C₁₀)aryl(C₁-C₆)alkylene; and
R⁸ is C₃-C₈ alkylene.

2. The balloon of claim 1, wherein x is from 0.02 to 0.15.

3. The balloon of claim 1, wherein m is from 0.10 to 0.75 and p is from 0.10 to 0.75.

4. The balloon of claim 1, wherein m is from 0.25 to 0.35, p is from 0.40 to 0.50, m+p is from 0.70 to 0.80, q is from 0.15 to 0.25, and x is from 0.03 to 0.10.

5. The balloon of claim 1, wherein the ratio of q to x is from 5:1 to 1:1.

6. The balloon of claim 1, wherein the ratio of q to x is from 4:1 to 2:1.

7. The balloon of claim 1, wherein R³ and R⁴ are hydrogen, (C₁-C₆)alkyl, CH₃—CH₂—CH(CH₃)—, (CH₃)₂CH—CH₂—, Ph-CH₂—, or (CH₃)₂CH—.

8. The balloon of claim 1, wherein R⁶ is according to Formula II.

9. The balloon of claim 1, wherein R⁷ is C₆aryl-CH₂—.

10. The balloon of claim 1, wherein the microparticles comprise from 35 to 45 wt % of rapamycin.

11. A method of forming microparticles comprising the steps of:
a. forming an oil phase comprising from 1 to 2.5 wt % Rapamycin, 1.5 to 2.5 wt % of a random copolymer according to Formula I, and from 95 to 97.5 wt % of chloroform or dichloromethane,
b. forming a continuous phase comprising water and from 0 to 1 wt % of an emulsifier, and
c. adding the oil phase to continuous phase under high shear,
wherein the random copolymer according to Formula I is as follows: 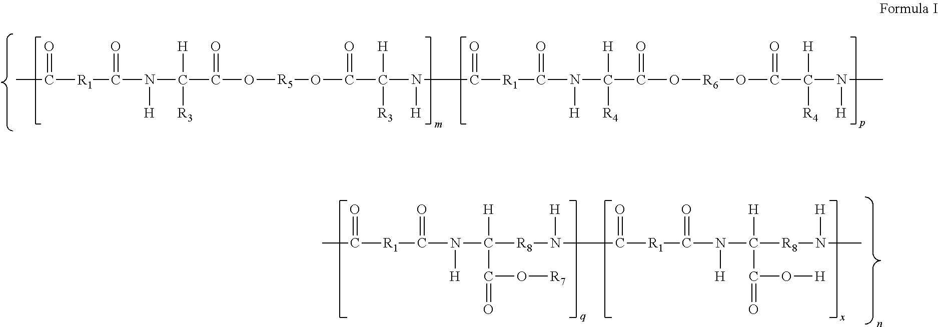
wherein
m, p, q, and x represent the equivalents of the corresponding units in the random copolymer and m+p+q+x=1;
m is from 0 to 0.75, p is from 0 to 0.75, m+p is from is from 0.5 to 0.9, q is from 0.05 to 0.25, x is from 0.03 to 0.25, and q:x is from 9:1 to 1:4;
n is from 5 to 300;
R¹ is C₂-C₂₀ alkylene;
R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—, CH═C—CH₂—, (CH₃)₂CH—, Ph-NH—, 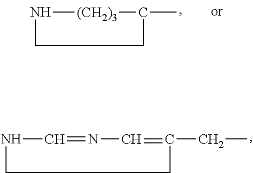
wherein R³ and R⁴ may be the same or different;
R⁶ is according to Formula II or Formula III; 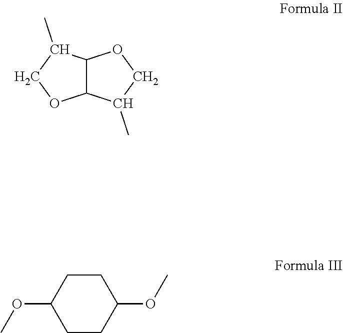
R⁷ is (C₆-C₁₀)aryl(C₁-C₆)alkylene; and
R⁸ is C₃-C₈ alkylene.

12. The method according to claim 11, wherein the oil phase further comprises an antioxidant.

13. The method according to claim 12, wherein the antioxidant comprises butylated hydroxytoluene.

14. The method according to claim 11, wherein the emulsifier comprises a polyvinyl alcohol.

15. The method according to claim 11, wherein the oil-phase comprises from 1.25 to 2.1 wt % of Rapamycin, from 1.5 to 2.5 wt % of the random copolymer according to Formula I, and from 95.5 to 97.2 wt % of chloroform or dichloromethane.

16. The method according to claim 11, wherein the oil-phase comprises from 1.25 to 2.1 wt % of Rapamycin, from 1.9 to 2.07 wt % of the random copolymer according to Formula I, and from 95.5 to 97.0 wt % of chloroform or dichloromethane.

17. A dispersion of microparticles in water formed by the method of claim 1.

18. The dispersion of claim 17, wherein the microparticles have a Dv90 particle size of from 1 to 11 μm, as measured with static laser scattering, and comprise from 35 to 45 wt % of rapamycin.

19. A plurality of microparticles having a Dv90 particle size of from 1 to 11 μm, as measured with static laser scattering, the microparticles consisting of from 35 to 50 wt % of rapamycin, based on the total weight of the microparticles, and a random copolymer according to Formula I: 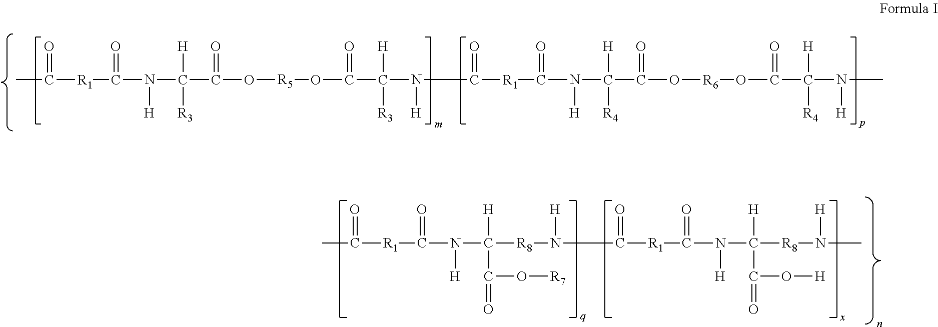
wherein
m, p, q, and x represent the equivalents of the corresponding units in the random copolymer and m+p+q+x=1;
m is from 0 to 0.75, p is from 0 to 0.75, m+p is from is from 0.5 to 0.9, q is from 0.05 to 0.25, x is from 0.01 to 0.25, and q:x is from 9:1 to 1:4;
n is from 5 to 300;
R¹ is C₂-C₂₀ alkylene;
R³ and R⁴ are hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃+, —(CH₂)₃NHC(═NH₂+)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph-CH₂—, CH═C—CH₂—, (CH₃)₂CH—, Ph-NH—, 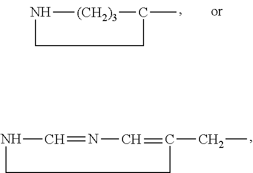
wherein R³ and R⁴ may be the same or different;
R⁶ is according to Formula II or Formula III; 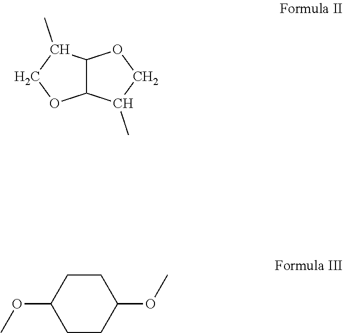
R⁷ is (C₆-C₁₀)aryl(C₁-C₆)alkylene; and
R⁸ is C₃-C₈ alkylene.

20. A coating comprising the plurality of microparticles according to claim 19.

21. A method of treating a human or animal patient comprising the steps of:
a. providing the balloon in accordance with claim 1;
b. positioning the balloon in a blood vessel of a patient;
c. inflating the balloon and thereby transferring a quantity of the microparticles to a wall of the blood vessel; and
d. deflating the balloon and removing the coated balloon from the blood vessel of the patient.

22. Use of the balloon according to claim 1 for treating an arterial disease.

23. The balloon according to claim 1 for use in treating an arterial disease of a patient.
